(19) United States
(12) Patent Application Publication
(10) Pub. No.: US 2012/0283553 A1
(43) Pub. Date: Nov. 8, 2012

(54) Echogenically Enhanced Device

(76) Inventors: Edward H. Cully, Flagstaff, AZ (US); Keith M. Flury, Flagstaff, AZ (US)

(21) Appl. No.: 13/465,367

(22) Filed: May 7, 2012

Related U.S. Application Data
(60) Provisional application No. 61/483,094, filed on May 6, 2011.

(51) Int. Cl. A61B 8/14

(52) U.S. Cl. 600/424

(57) ABSTRACT

Devices with enhanced visualization in ultrasound imaging are provided.

### CROSS REFERENCE TO RELATED APPLICATIONS

This application claims priority to provisional application Ser. No. 61/483,094, filed May 6, 2011.

### FIELD OF THE INVENTION

The present invention relates to devices with enhanced echogenicity for better visualization in ultrasound imaging and methods for enhancing echogenicity of a device.

### BACKGROUND OF THE INVENTION

Ultrasound technology has advantages over other imaging modalities. Along with the health advantage of reducing or eliminating exposure to x-rays (fluoroscopy), the equipment needed is small enough to move and it has advantages in diagnosing sub-surface tissue morphology. Furthermore, ultrasound transducers can be made small enough to place inside the body where they can provide better resolution than is currently available with magnetic resonance imaging and x-ray computed tomography. Further, device enhancements which increase their echogenicity to accommodate ultrasound enable clinicians to quickly and properly treat patients, saving time and money.

Many interventional tools and instruments are designed with polished surfaces that render the devices virtually invisible on ultrasound. Interventional tools and instruments are herein referred to as “device(s)”. The present invention relates to a device enhancement to increase echogenicity of interventional devices. Interventional devices include, but are not limited to, septal puncture needles as well as implantable devices, such as, but not limited to, stents, filters, stent graphs, and/or heart valves.

Ultrasound image device enhancement or “echogenicity” has been studied for many years. When sound waves contact a smooth surface, the angle of incidence and reflection are the same. If the object is located at a steep angle most or all the sound waves bounce away from a transmitting/receiver source. With such steep angles, even highly reflective devices can be invisible by ultrasound if scattering does not direct sound back to a source transducer. Conversely, if an object is perpendicular, the sound waves reflecting directly back may cause a “white out” effect and prevent the operator from seeing around the object. This affect is referred to as specular reflection.

Medical device manufacturers have tried a variety of techniques to improve visibility of devices to ultrasound. Examples include roughening the surface of the device, entrapping gas, adhering particles to substrate surfaces, creating indentations or holes in the substrates and using dissimilar materials.

### SUMMARY OF THE INVENTION

An aspect of the present invention relates to an echogenically enhanced interventional tool or device. The interventional tool or device to be imaged ultrasonically has a surface with one or more apertures and a polymeric film in close contact with the surface of the tool or device which covers at least a portion of the one or more apertures.

Another aspect of the present invention relates to a method for enhancing echogenicity of an interventional. tool or device. In this method, one or more apertures are made in a surface of an interventional tool or device. A polymeric film is then placed in close contact with the surface covering at least a portion of the one or more apertures.

### BRIEF DESCRIPTION OF THE FIGURES

FIG. 1 shows an interventional tool or device with a plurality of apertures in its surface.

FIGS. 2A and 2B show the same interventional tool or device of FIG. 1 with a polymeric film in close contact with the surface of the device so that the apertures are closed.

FIG. 3 is a bar graph showing results of a comparison of the dB increase above control of a device of the present invention with a polymeric film covering apertures in the surface of the device as depicted in FIGS. 2A and 2B and another commercially available coated device.

FIG. 4 is a plot of the reflected energy at various angles, which reflects increased echogenic response.

### DETAILED DESCRIPTION OF THE INVENTION

The present invention relates to an enhancement to increase echogenicity of these interventional devices. The echogenically enhanced device of the present invention comprises a device to be imaged ultrasonically having a surface with one or more apertures. The interventional device of the present invention further comprises a polymeric film in close contact with the surface of the device which covers at least a portion of the one or more apertures.

Examples of interventional tools or devices which can be enhanced visually in ultrasound imaging in accordance with the present invention include, but are not limited to, medical devices such as permanent implantable or temporary indwelling devices, such as catheters, guide wires, stents and other accessories and tools, surgical instruments, and needles such as septal puncture needles. However, as will be understood by the skilled artisan upon reading this disclosure, the techniques described herein for visually enhancing a device via ultrasound imaging are adaptable to many different fields and devices.

In accordance with the present invention, one or more apertures are made in a surface of the interventional tool or device. The apertures of the present invention may be divots in the surface of an otherwise smooth device surface, or holes through the surface of the device, or grooves formed in the device surface, or any other topographical asperities in the otherwise smooth surface of the device.

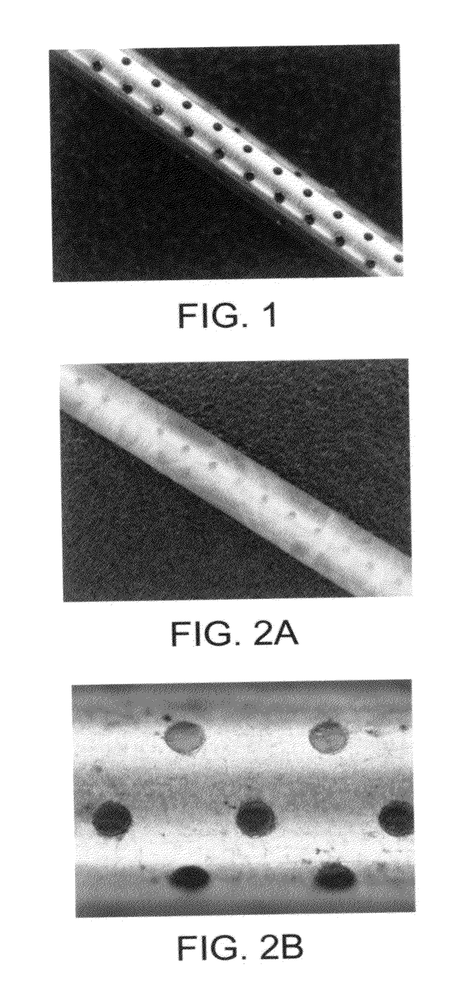

In one embodiment, as depicted in FIG. 1, a plurality of apertures is made in the surface of the interventional tool or device.

In one embodiment, in addition to apertures in the surface of the interventional device, the surface is also roughened. In one embodiment, the surface roughness of the device has an average surface roughness of less than 1 μm.

In embodiments wherein the polymeric film is bonded to the device, surface roughening may be useful to increase adhesion.

Echogenicity of this device is enhanced in accordance with the present invention by positioning an echogenic polymeric film in close contact with the surface of the device to cover at least a portion of the aperture or apertures in the surface of the interventional tool or device. In one embodiment, the polymeric film covers the entire aperture or apertures in the surface of the interventional tool or device. In one embodiment, the polymeric film surrounds the entire surface of the interventional tool or device. The polymeric film covering may also restore luminal competency to a medical device (needle, biopsy punch, etc) in which through-holes/apertures have been added. n the case of divots or grooves, the polymeric film covering, especially the ePTFE film, may restore surface smoothness, which is preferable in most endoluminal procedures.

In some embodiments of the present invention, the echogenic response of the device may be adjustable. One adjustable embodiment comprises a hollow device with through-hole apertures in the surface covered by a thin polymeric film. The pressure within the device can be increased or decreased to change the resonant characteristic of the polymeric film covering said apertures so as to produce a change in the device's echogenic response while viewed via ultrasound. In another embodiment, the tension of the polymeric film covering the apertures of a device may be adjustable. By increasing or decreasing the tension of this polymeric film, the echogenicity of the device can be adjusted. The shape of the apertures can be varied to change the echogenicity that is achieved.

Any biocompatible polymeric film capable of an echogenic response with minimal profile impact can be used. In one embodiment, the polymeric film comprises a microporous fluoropolymer such as expanded polytetrafluoroethylene (PTFE). In another embodiment, the polymeric film may be a thin polyolefin film which may or may not be porous. The different thickness of material will change the topography when the sleeve is “activated.” Different topography will change the echogenicity of the object. The thickness of said polymeric films should be less than 0.010″. In another embodiment, said polymeric film thickness is less than 0.006″. In another embodiment, said polymeric film thickness is less than 0.003.

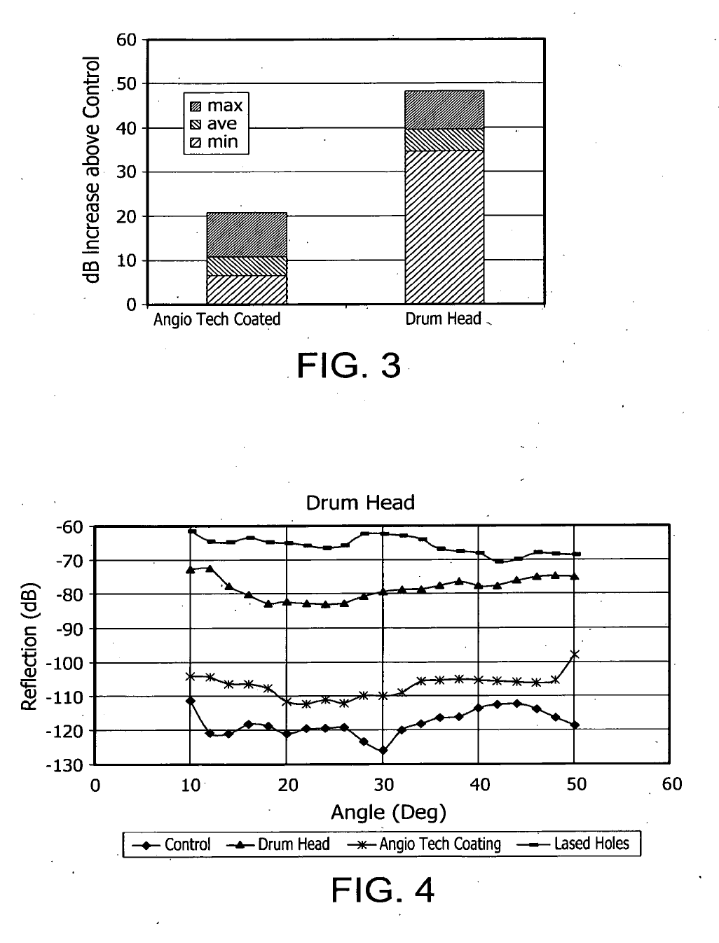

Enhanced echogenicity of a device of the present invention was demonstrated experimentally. Results are depicted in FIG. 3 which shows a comparison of the dB increase above control of a device of the present invention and an Angiotech coated device.

The following non-limiting examples are provided to further illustrate the present invention.

### EXAMPLES

### Example 1

### Materials

A stainless steel needle with the dimensions of 0.040″ diameter and approximately 4.8″ long was used as the test article for echogenic enhancement. An unmodified needle was used as control to compare the results of the modification. Echogenicity of a stainless steel needle with a plurality of apertures covered by a polymeric film in accordance with the present invention was also compared to an Angiotech coated needle (Angiotech Pharmaceuticals, Inc., 1618 Station Street, Vancouver, BC Canada V6A 1B6). The apertures are staggered 45° 0.178 mm in diameter and spaced 0.38 mm apart.

### Example 2

### Methods

Three different methods were used to evaluate and compare the treated samples.

All samples were subjected to an acoustic wave imaging system. The testing apparatus consisted of a 7.5 MHz transmitting/receiving transducer mounted onto a flat bar with a sample holder placed approximately 2.5 cm at the transducer's focal length. The 7.5 MHz transducer produced a wave length (λ) of 200 microns. At 2.5 cm the width of the signal was approximately 1 mm. The needle sample was placed into a holder that is perpendicular to the axis of the emitting transducer. This is 0 degrees. The sample holder is removable for ease of changing out the sample. The holder is magnetically held in a rotatable goniometer for measuring the angle of the sample relative to the transmitting and receiving transducer. The sample and transducer were submerged into a room temperature water tank. Before collecting the data, every sample was aligned with the transducer. This was accomplished by increasing the attenuation setting on the pulser/receiver controller (approximately 40 dB) to prevent saturation of the received signal. The operator then visually monitored the wave signal while manually rotating the goniometer and dialing the fine adjustment knobs on the transducer to achieve a maximum return signal. The attenuation was adjusted to a reference point of approximately 1 volt. The attenuation setting and the goniometer indication were recorded. The goniometer was rotated 10 degrees from the recorded indication. Since the signal typically decreases off of perpendicular (specular reading) the attenuation was reduced. The reduced level allowed a strong enough signal during collection, without saturation of the receiver. The sample was rotated through the entire angular rotation to ensure that the signal did not saturate or significantly move away from or closer to the transducer moving the signal out of the data collection window. Significant time shift was an indication that the transducer was not aligned with the center or pivot of the sample. Once the set-up was completed, the goniometer was moved to the 10 degree mark and the collection of points was taken to 50 degrees at 2 degree increments. Equipment connected to the transducer and test fixture measured reflection. The software, Lab View, and hardware were used for data collection and later analysis.

A second evaluation of samples was performed in a silicone phantom submersible in a blood substitute from ATS laboratories to increase attenuation and create a more realistic image environment. Using a 6.5 mHz transducer ultrasound system, the samples were inserted into the phantom. A still image was captured for each sample. These images were visually compared to control images and inspected for consistency with the transducer 2D data. The data was collected at three different times. Between collections two and three the transducer was rebuilt. Thus, while the absolute dB scale of plots is not the same, the relative deltas are of importance.

The third evaluation was a surface analysis using an optical comparator, Veeco Model NT3300. All raw data was further processed by the machine software to better evaluate the samples. The macroscopic tilt and cylindrical curvature were removed. A Gaussian filter (Fourier) was selected to filter frequencies below 20⁻¹ mm. Incomplete interior points were restored with a maximum of 3 or 5 pixels. All samples were masked at the edges to remove large data drop out sections and anomalies associated with the filtering. 2D samples were processed first followed by 3D samples.

Total roughness height, Rt or PV, which is the maximum peak to valley height of the surface profile within the assessment length, was used to depict the surface characteristics.

A comparison of the dB increase above control of a device of the present invention and an Angiotech coated device is depicted in FIG. 3.

What is claimed is:

1. An echogenically enhanced interventional device comprising:
(a) an interventional device to be imaged ultrasonically, said tool or device having a surface with one or more apertures; and
(b) a polymeric film in close contact with the surface of said device which covers at least a portion of the one or more apertures.

2. The echogenically enhanced interventional device of claim 1 wherein the entire one or more apertures are covered by the polymeric film.

3. The echogenically enhanced interventional device of claim 1 wherein the surface of said device comprises a plurality of apertures.

4. The echogenically enhanced interventional device of claim 1 wherein the polymeric film surrounds the surface of said device.

5. The echogenically enhanced interventional device, of claim 1 wherein the tension on the polymeric film surrounding the surface of said device is adjustable.

6. The echogenically enhanced interventional device of claim 1 wherein the polymeric film comprises a microporous fluoropolymer.

7. The echogenically enhanced interventional device of claim 1 wherein the polymeric film comprises expanded polytetrafluoroethylene (PTFE).

8. The echogenically enhanced interventional device of claim 1 wherein the interventional device is a surgical instrument.

9. The echogenically enhanced interventional device of claim 1 wherein the interventional device is a septal puncture needle.

10. The echogenically enhanced interventional device of claim 1 wherein the surface of the interventional device is roughened.

11. The echogenically enhanced interventional device of claim 9 wherein the surface is roughened less than 1 μm.

12. A method for enhancing echogenicity of an interventional device, said method comprising:
producing one or more apertures in a surface of an interventional device; and
positioning a polymeric film in close contact with the surface so that at least a portion of the one or more apertures is closed.

13. The method of claim 12 wherein the polymeric film is positioned to entirely close the one or more apertures in the interventional tool or device.
